(19) United States
(12) Patent Application Publication
(10) Pub. No.: US 2025/0268510 A1
(43) Pub. Date: Aug. 28, 2025

(54) METHOD AND TOOL FOR PREDICTING LANGUAGE DEVELOPMENT AND COMMUNICATION CAPABILITIES OF INFANT AND TODDLER

(71) Applicant: The Chinese University of Hong Kong, HONG KONG (CN)

(72) Inventors: Patrick Chun Man WONG, Hong Kong (CN); Nikolay NOVITSKIY, Hong Kong (CN); Gangyi FENG, Hong Kong (CN); Ting Fan LEUNG, Hong Kong (CN); Hugh Simon Hung San LAM, Hong Kong (CN); Akshay Raj MAGGU, Morrisville, NC (US)

(21) Appl. No.: 18/097,578

(22) PCT Filed: Jul. 16, 2021
(86) PCT No.: PCT/CN2021/106684

§ 371 Date: Jan. 17, 2023

(30) Foreign Application Priority Data
Jul. 17, 2020 (CN) 202010692253.5

(51) Int. Cl. A61B 5/38; A61B 5/246; A61B 5/372

(52) U.S. Cl. CPC A61B 5/38; A61B 5/246; A61B 5/372; A61B 2503/04

(57) ABSTRACT

A method for predicting a development level difference of language and communication capability normality of a healthy infant or toddler. The method comprises obtaining electroencephalogram (EEG) or magnetoencephalogram (MEG) waveform data of a healthy infant or toddler caused by external auditory stimulus; obtaining, from the EEG or MEG waveform data, quantitative data that represents measurement index data of a central nervous system caused by the external auditory stimulus; and using a data set to train a machine learning classifier, so as to obtain a prediction result. Further provided are a relevant tool and an integrated system.

### CROSS-REFERENCE TO RELATED APPLICATIONS

This application is a national stage of International Application No. PCT/CN2021/106684, filed on Jul. 16, 2021, which claims priority to Chinese Patent Application No. 202010692253.5 filed on Jul. 17, 2020. Both of the aforementioned applications are hereby incorporated by reference in their entireties.

### TECHNICAL FIELD

The present application generally relates to a merged field of medical and artificial intelligence. In particular, the present application provides methods, tools, and systems based on electroencephalogram (EEG) or magnetoencephalogram (MEG) examination and artificial intelligence training to forecast normal developmental differences of language and communication capabilities of healthy infants or toddlers.

### BACKGROUND

Language and communication is fundamental to human development and affects all aspects of later childhood and adulthood. It has been reported that language and communication capability not only influences academic success for toddlers, but is also a significant predictor of employment and wages later in life. It will be advantageous for the development of a subject if its normal development of language and communication capabilities can be forecasted in early life, and necessary active interventions and training can be performed based on the forecasting.

### SUMMARY

In a first aspect, the present application provides a method of forecasting a normal developmental difference of language and communication capability of a healthy infant or toddler, comprising
obtaining electroencephalogram (EEG) or magnetoencephalogram (MEG) waveform data from the healthy infant or toddler in response to an external auditory stimulus;
extracting quantitative data from the EEG or MEG waveform data, wherein the quantitative data include measurement index data characterizing the central nervous system's response to the external auditory stimulus;
analyzing the quantitative data using a machine learning classifier, wherein the machine learning classifier has been trained to provide a forecasting score for forecasting a normal developmental difference of language and communication capability of a population to which the healthy infant or toddler belongs, and wherein the training is based on corresponding quantitative data from a training data set obtained from a plurality of healthy infants or toddlers known to have normal development of language and communication capability; and
generating, based on an output from the machine learning classifier, a forecasting score of the normal developmental difference of language and communication capability of the healthy infant or toddler.

In some embodiments, the method further comprises obtaining EEG or MEG waveform data of the healthy infant or toddler at rest; and extracting corresponding quantitative data from the EEG or MEG waveform of the healthy infant or toddler at rest, wherein the quantitative data comprises measurement index data of the central nervous system at rest.

In some embodiments, the external auditory stimulus comprises a plurality of different stimuli, and the quantitative data include measurement index data of the central nervous system's response to one or more of the plurality of external auditory stimuli and measurement index data of the central nervous system at rest.

In some embodiments, the quantitative data include data characterizing functional activity of the processing pathway associated with the auditory center.

In some embodiments, the quantitative data include data characterizing functional activity of the inferior colliculus or centers connected to the inferior colliculus.

In some embodiments, the quantitative data include data characterizing functional activity of the primary auditory cortex, including the Heschl's Gyrus, or centers connected to the Heschl's Gyrus.

In some embodiments, the external auditory stimulus is from a language and speech signal in which pitch patterns are used to convey meaning at the vocabulary or lexical level. In some embodiments, the external auditory stimulus is from Chinese including Mandarin and Cantonese.

In some embodiments, the external auditory stimulus is from a language and speech signal in which pitch patterns are used to convey meaning at the phrasal or sentential level. In some embodiments, the external auditory stimulus is from Chinese including Mandarin and Cantonese.

In some embodiments, the external auditory stimulus is a Chinese pinyin with one or more tones.

In some embodiments, the EEG or MEG is performed while the healthy infant or toddler is in natural sleep or in awake state.

In some embodiments, the healthy infant or toddler has an age of 18 months or less. In some embodiments, regarding the generation of the training data set, when the training data set is obtained from the plurality of healthy infants or toddlers, the plurality of healthy infants or toddlers have ages of 18 months or less.

In some embodiments, the machine learning classifier is a support vector machine (SVM) and the forecasted normal developmental difference of language and communication capability is a high-degree or a low-degree assessment, or a continuous performance assessment.

In some embodiments, the machine learning classifier is a support vector regression algorithm (SVR) or ranking support vector machine (RankSVM) and the forecasted normal developmental difference of language and communication capability is quantified.

In some embodiments, analyzing the quantitative data further comprises analyzing one or more variables associated with a normal developmental difference of language and communication capability, such as gender and birth data.

In some embodiments, quantitative data are extracted from the EEG or MEG waveform by a method selected from the group consisting of Automatic peak detection, Fast Fourier Transform, Autocorrelation, Root-Mean-Square (RMS), Morlet Wavelet Transform, Discrete Wavelet Transform, Wavelet Scattering, Stimulus-Response Cross-correlation, Empirical Mode Decomposition, and Hilbert-Huang Transform.

In some embodiments, the quantitative data include one or more selected from the group consisting of time-domain peak amplitude, time-domain peak latency, fundamental frequency (F0), harmonics, signal-to-noise ratio, RMS amplitude, correlation coefficient, inter-trial phase coherence, phase-locking coefficient, response consistency, pitch strength, pitch error, and pitch-tracking accuracy.

In some embodiments, extracting quantitative data from the EEG waveform comprises segmenting the EEG waveform by stimulus onset markers, and transforming each segment to frequency domain with Fast Fourier Transform (FFT) in a sliding time window with an overlap between the windows. In some embodiments, the quantitative data include a matrix represented by T*(E*3)*F array for the healthy infant or toddler, wherein T is the number of the time windows, E is the number of segments for each stimulus (e.g. a Chinese pinyin with one tone) and F is the number of frequency bins from the FFT analysis, and wherein the (E*3)*F matrix for each T is normalized first within rows and then within columns, thereby removing an effect of the absolute amplitude of the spectrum and leaving only the frequency-dependent patterns over time. In some embodiments, the machine learning classifier is a support vector machine (SVM) using parameters including gaussian kernel, C and gamma.

In some embodiments, a classification made by the machine learning classifier is subjected to cross-validation, and the outcome of the cross-validation is average accuracy, specificity, sensitivity, Area Under Curve (AUC), parity rate, correlation coefficient or a combination thereof across certain folds (e.g. 5, 10 or more folds).

In a second aspect, the present application provides a computer-readable medium storing a plurality of instructions that, when executed by a processor of a computer system, control the computer system to perform a plurality of operations to implement the methods described in the first aspect.

In a third aspect, the present application provides a computer system comprising the computer readable medium described in the second aspect; and
one or more processors for executing instructions stored on the computer-readable medium.

In a fourth aspect, the present application provides a computer system comprising a memory; and a processor communicatively coupled to the memory and configured to perform a plurality of operations to implement the methods described in the first aspect.

In a fifth aspect, the present application provides a system for forecasting a normal developmental difference of language and communication capability of a healthy infant or toddler, including a plurality of means for implementing the methods described in the first aspect.

In a sixth aspect, the present application provides a method of generating a machine learning classifier for forecasting a normal developmental difference of language and communication capability of a healthy infant or toddler, comprising obtaining electroencephalogram (EEG) or magnetoencephalogram (MEG) waveform data from one or more healthy infants or toddlers in response to an external auditory stimulus, and extracting quantitative data from the EEG or MEG waveform data, wherein the quantitative data include measurement index data characterizing the central nervous system's response to the external auditory stimulus; after a certain period of time (e.g., 6 to 12 months), performing a language and communication capability test on the one or more healthy infants or toddlers, and obtaining qualitative or quantitative data indicative of its/their language and communication capability; and inputting the quantitative data extracted from the EEG or MEG waveform data and the qualitative or quantitative data indicative of the language and communication capability into a machine learning classifier for training to generate a machine learning classifier for forecasting a normal developmental difference of language and communication capability of a healthy infant or toddler.

### BRIEF DESCRIPTION OF THE DRAWINGS

FIG. 1 shows a diagram of obtaining auditory-evoked EEG data from an infant, including placing electrodes on the head of the infant, and presenting auditory stimuli via insert earphones while the infant is asleep (or in awake state).

FIG. 2 shows the EEG responses to various Chinese tones according to exemplary embodiments of the present application. As external auditory stimuli, three tones were imbedded into the syllable /ga/: Cantonese tone /ga2/, Mandarin tone /ga3/and Cantonese tone /ga4/. (Note: the infants were being raised in Cantonese-speaking homes by Cantonese-speaking caregivers). The early-latency EEG responses faithfully reflect the time course and the spectral composition of the auditory stimulus. Panel A shows the time courses of the auditory stimuli (left column) and averaged EEG responses (middle column) as well as the averaged spectrogram of the EEG response (right column). The spectral amplitude density of the EEG response concentrates within the upper quartile of the auditory stimulus spectral density. Panel B shows the spectrogram of the auditory stimulus (left column) and averaged EEG spectrograms of the infants at 1, 3, 6, 9 and 15 months of age in response to the auditory stimuli. The older the infants, the clearer is the overlap between the auditory and EEG spectral structures.

FIG. 3 shows that, by application of the support vector machine (SVM) classifier to the EEG responses of the infants at different months of age to the three Chinese lexical tones /ga2/ /ga3/ and /ga4/to generate forecasted classification outcomes, ability of the infant brain to discriminate the tones can be measured. The SVM classifier was fed with the Fourier transforms of the consequent 50-ms intervals of the along the time course of the EEG data response to the three tones. The classification was cross-validated by training on the 9/10th of the data and testing on the remaining 1/10th of the data for each of the 10 folds of the randomly split the data. Chance level of classification accuracy for 3 tones is 33%. The figure illustrates that the classification accuracy has two peaks along the time course of the EEG response and that the accuracy grows with the age of the infant. The current figure illustrates classification across subjects. The same procedure was performed within each individual subject.

FIG. 4 shows that two language and communication outcome measures (Later Gestures and Interactive Acts derived from the Chinese Communication Development Inventories) were forecasted with an accuracy of higher than 80% by a machine-learning model (SVM classifier) trained and cross-validated with EEG data taken at the age of about 6 months. The figure shows the bootstrapped distribution (10000 times) of the classification accuracy for attributing the subject's two language and communication outcome measures at 15 months of age to the lower or upper-half performers (median split). The classification was performed for both real (left) and permuted subject labels (right). The mean of the permuted-label classification accuracy is at the chance level of 50%. The mean of real-label classifications is in both cases higher than 95% of the permuted-label classification (vertical dashed line) indicating that classification was significantly higher than chance (p<0.05).

### DETAILED DESCRIPTION OF THE EMBODIMENTS

As mentioned previously, language and communication skills are very important qualities for subjects and have a significant impact on academic and career aspects. Chinese and English are the two most commonly used languages in the world, but they have markedly different linguistic structures. One prominent difference is the use of lexical tone, or lexically meaningful pitch patterns, in Chinese, while in English or many other languages, pitch patterns are often used to express sentential meanings. For example, for a syllable, Mandarin Chinese typically has four tones, while in some Chinese dialects, there can be more tones (e.g., Cantonese may include six tones). As such, the ability of human nervous system to encode pitch accurately is critical for language development. Research has found that the auditory neural system in Chinese-learning infants has the remarkable ability to track pitch.

One of the problems to be solved by the present application is to forecast a normal developmental difference of language and communication capability of subjects in their infant or toddler period. Based on the forecasting result, specialized active interventions and training can be made if necessary. To solve such a technical problem, the present application provides a technical platform for forecasting the developmental trend of future language and communication capability of infants and toddler mainly by means of their neural data (such as data extracted from EEG or MEG). Machine learning techniques are used to train classifiers to make such forecasting. In order to obtain the training data set required for training the classifiers, a certain amount of neural data samples from infants is required. Then, subjects are evaluated for their language and communication capabilities, and two parts of results are subjected to cross-validation and training. Once the training is complete, a machine learning classifier can be used to forecast normal developmental difference of language and communication capability of other infants.

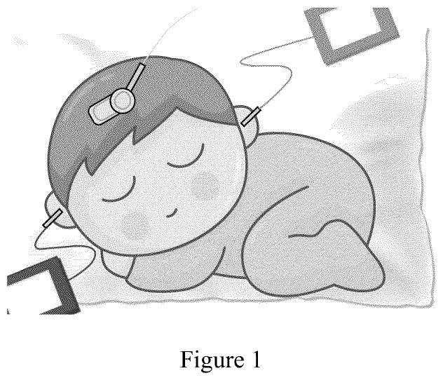

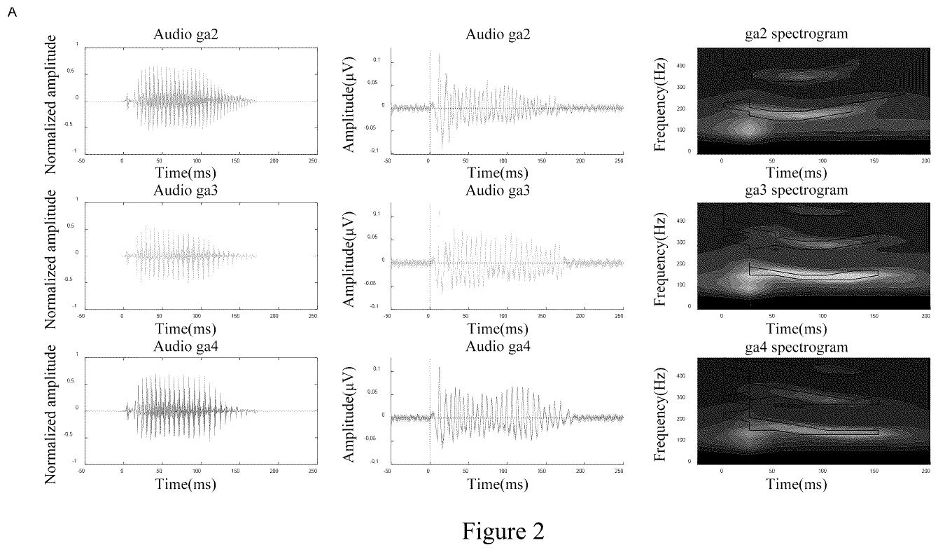

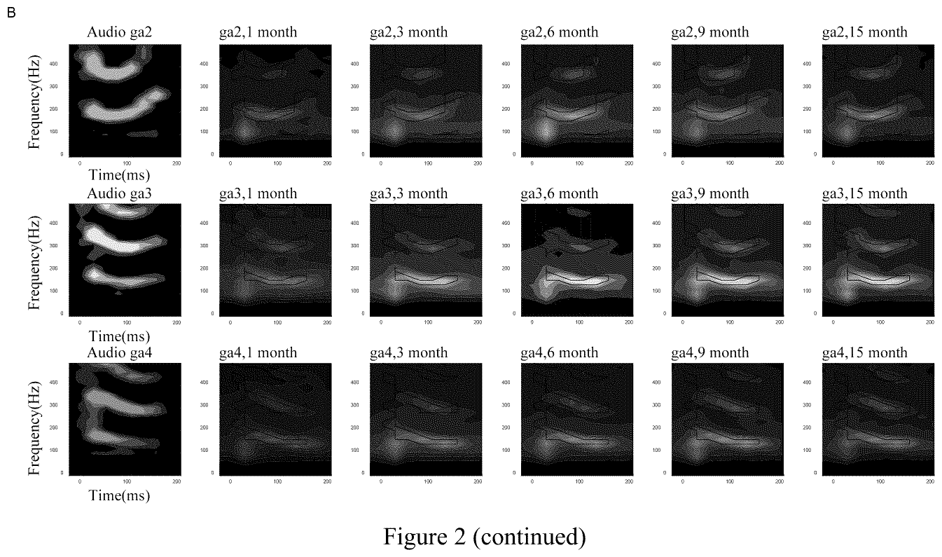

In general, the method of the present application may involve placing surface electrodes around an infant or toddler's head while EEG recordings are made. The infant or toddler hears auditory stimuli in natural sleep (FIG. 1). The method may involve measuring neural responses from the subcortical auditory brainstem and specifically how an infant or toddler's auditory brainstem follows the frequency (pitch) of the stimulus (see FIG. 2 for exemplary results), thereby using the ability to observe pitch tracking in the subcortical auditory brainstem and connected neural structures to forecast an infant or toddler's future language and communication development.

Thus, in a first aspect, the present application provides a method of forecasting a normal developmental difference of language and communication capability of a healthy infant or toddler, comprising:
obtaining electroencephalogram (EEG) or magnetoencephalogram (MEG) waveform data from the healthy infant or toddler in response to an external auditory stimulus;
extracting quantitative data from the EEG or MEG waveform data, wherein the quantitative data include measurement index data characterizing the central nervous system's response to the external auditory stimulus;
analyzing the quantitative data using a machine learning classifier, wherein the machine learning classifier has been trained to provide a forecasting score for forecasting a normal developmental difference of language and communication capability of a population to which the healthy infant or toddler belongs, and wherein the training is based on corresponding quantitative data from a training data set obtained from a plurality of healthy infants or toddlers known to have normal development of language and communication capability; and
generating, based on an output from the machine learning classifier, a forecasting score of the normal developmental difference of language and communication capability of the healthy infant or toddler.

In some embodiments, the method further comprises obtaining EEG or MEG waveform data of the healthy infant or toddler at rest; and extracting corresponding quantitative data from the EEG or MEG waveform of the healthy infant or toddler at rest, wherein the quantitative data comprises measurement index data of the central nervous system at rest.

In some embodiments, the EEG or MEG is performed while the healthy infant or toddler is in natural sleep or in awake state. In some embodiments, the healthy infant or toddler has an age of 18 months or less. In some embodiments, regarding the generation of the training data set, when the training data set is obtained from the plurality of healthy infants or toddlers, the plurality of healthy infants or toddlers have ages of 18 months or less.

As used herein, “healthy” refers to a state of health at least in terms of listening, speaking, and intelligence, which are in relation to language and communication capability, and includes, but is not limited to, a state of complete health.

As used herein, “infant” and “toddler” are defined according to the usual age criteria, i.e., an age of 0-12 months for infants and an age of 1 to 3 years for toddlers.

As used herein, “external auditory stimulus” refers to an auditory stimulus in terms of language, and may be selected depending on the structure and composition of different languages. It will be appreciated that the language of an external auditory stimuli should match the language to be learnt and used by the test subject in its future growth. Generally, a single external auditory stimulus can be a relatively simple language unit, e.g., a letter/pinyin, character/word (different tones), and phrase. In some embodiments, an external auditory stimulus is from a language and speech signal in which pitch patterns are used to convey meaning at the vocabulary or lexical level. In some embodiments, an external auditory stimulus is from Chinese including Mandarin and Cantonese. In some embodiments, an external auditory stimulus is from a language and speech signal in which pitch patterns are used to convey meaning at the phrasal or sentential level. In some embodiments, an external auditory stimulus is from Chinese including Mandarin and Cantonese. Although the exemplary language, on which the present application is carried, is Chinese (Chinese, in which pitch patterns are used to convey meaning at the vocabulary and lexical level, is particularly applicable to the present application), the applications of the inventions of the present application are not limited to Chinese. Other languages, such as English, French, German, Spanish, Portuguese, Japanese, and Korean, are also applicable to the present application, since, in almost all languages, pitch patterns are at least used to convey meaning at the vocabulary or lexical level.

In some embodiments, the external auditory stimuli comprise a plurality of different stimuli. In some embodiments, the quantitative data include measurement index data of the central nervous system's response to one or more of the plurality of external auditory stimuli and measurement index data of the central nervous system at rest. As a non-limiting example, the plurality of external auditory stimuli may be a Chinese pinyin with a plurality of tones.

After obtaining electroencephalogram (EEG) or magnetoencephalogram (MEG) waveforms, data characterizing the central nervous system's response to external auditory stimuli need to be extracted from the waveforms. For example, quantitative data may include data characterizing functional activity of the processing pathway associated with the auditory center; or data characterizing functional activity of the inferior colliculus or centers connected to the inferior colliculus; or data characterizing functional activity of the primary auditory cortex, including the Heschl's Gyrus, or centers connected to the Heschl's Gyrus.

There are a number of methods available for extracting the quantitative data from EEG or MEG waveforms, including, but not limited to, Automatic peak detection, Fast Fourier Transform, Autocorrelation, Root-Mean-Square (RMS), Morlet Wavelet Transform, Discrete Wavelet Transform, Wavelet Scattering, Stimulus-Response Cross-correlation, Empirical Mode Decomposition, Hilbert-Huang Transform, or a combination of the above.

There are a number of forms of quantitative data that can be extracted from EEG or MEG waveforms, including, but not limited to, time-domain peak amplitude, time-domain peak latency, fundamental frequency (F0), harmonics, signal-to-noise ratio, RMS amplitude, correlation coefficient, inter-trial phase coherence, phase-locking coefficient, response consistency, pitch strength, pitch error, pitch-tracking accuracy, or a combination of the above.

As a non-limiting example, the process of extracting quantitative data from the EEG waveform may comprise segmenting the EEG waveform by stimulus onset markers, and transforming each segment to frequency domain with Fast Fourier Transform (FFT) in a sliding time window with an overlap between the windows. In some embodiments, the quantitative data include a matrix represented by T*(E*3)*F array for the healthy infant or toddler, wherein T is the number of the time windows, E is the number of segments for each stimulus (e.g. a Chinese pinyin with one tone) and F is the number of frequency bins from the FFT analysis, and wherein the (E*3)*F matrix for each T is normalized first within rows and then within columns, thereby removing an effect of the absolute amplitude of the spectrum and leaving only the frequency-dependent patterns over time.

After obtaining the quantitative data, the quantitative data are analyzed using a machine learning classifier. The general working principle of machine learning classifiers is known. The machine learning classifier of the present application operates on a training data set derived from a plurality of similar subjects, who and the test subjects belong to the same population. As used herein, “same population” refers to a collection of subjects with the same linguistic and cultural background. For example, if a test subject grows in a Mandarin-speaking society, a Mandarin-based training data set should be used. Similarly, if a test subject grows in a Cantonese-speaking society, a Cantonese-based training data set should be used. The plurality of subjects used to create the training data set may have undergone the same external auditory stimuli and EEG/MEG quantitative data extraction as the test subjects, and the plurality of subjects may also subsequently (e.g., 6-12 months later) undergo an actual evaluation of language and communication capabilities, the results from which are used to create the training data set. There are a variety of tests for evaluating toddlers's language and communication capabilities, such as the Later Gestures and the Interactive Acts in the Chinese Communication Development Inventories.

In some embodiments, the machine learning classifier is a support vector machine (SVM) using parameters including gaussian kernel, C and gamma.

In some embodiments, the machine learning classifier is a support vector machine (SVM) and the forecasted normal developmental difference of language and communication capability is a high-degree or a low-degree assessment, or a continuous performance assessment.

In some embodiments, the machine learning classifier is a support vector regression algorithm (SVR) or ranking support vector machine (RankSVM) and the forecasted normal developmental difference of language and communication capability is quantified.

In some embodiments, a classification made by the machine learning classifier is subjected to cross-validation, and the outcome of the cross-validation is average accuracy, specificity, sensitivity, AUC, parity rate, correlation coefficient or a combination thereof across certain folds (e.g. 5, 10 or more folds).

In some embodiments, analyzing the quantitative data further comprises analyzing one or more variables associated with a normal developmental difference of language and communication capability, such as gender and birth data.

The Examples of the present application describe a specific embodiment of the method as described in the first aspect. It is to be understood that the features in the Examples may be applied to the above embodiments, if no technical contradiction.

In a second aspect, the present application provides a computer-readable medium storing a plurality of instructions that, when executed by a processor of a computer system, control the computer system to perform a plurality of operations to implement the methods described in the first aspect.

In a third aspect, the present application provides a computer system comprising:
the computer readable medium described in the second aspect; and
one or more processors for executing instructions stored on the computer-readable medium.

In a fourth aspect, the present application provides a computer system comprising:
a memory; and
a processor communicatively coupled to the memory and configured to perform a plurality of operations to implement the methods described in the first aspect.

In a fifth aspect, the present application provides a system for forecasting a normal developmental difference of language and communication capability of a healthy infant or toddler, including a plurality of means for implementing the methods described in the first aspect.

In a sixth aspect, the present application provides a method of generating a machine learning classifier for forecasting a normal developmental difference of language and communication capability of a healthy infant or toddler, comprising:
obtaining electroencephalogram (EEG) or magnetoencephalogram (MEG) waveform data from one or more healthy infants or toddlers in response to an external auditory stimulus, and extracting quantitative data from the EEG or MEG waveform data, wherein the quantitative data include measurement index data characterizing the central nervous system's response to the external auditory stimulus;
after a certain period of time (e.g., 6 to 12 months), performing a language and communication capability test on the one or more healthy infants or toddlers, and obtaining qualitative or quantitative data indicative of its/their language and communication capability; and
inputting the quantitative data extracted from the EEG or MEG waveform data and the qualitative or quantitative data indicative of the language and communication capability into a machine learning classifier for training to generate a machine learning classifier for forecasting a normal developmental difference of language and communication capability of a healthy infant or toddler.

Various technical solutions and technical features described in the first aspect are applicable to (maybe with certain adaptions) the second to sixth aspects, if no conflict. It should also be understood that the invention described in the sixth aspect involves the acquisition of quantitative data in EEG or MEG waveform data and the acquisition of qualitative or quantitative data to characterize language and communication capabilities. The implementation of the former acquisition may be referred to the method described in the first aspect. The implementation of the latter acquisition can be carried out in a number of ways, such as a number of modules related to language and communication skills in the Chinese Communication Development Inventories exemplified herein. Either qualitative or quantitative results can be applied, with appropriate data transformations, to machine learning classifiers for training.

It should be understood that, embodiments of the present application can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of the embodiments of the present application. However, other embodiments of the present application may relate to specific embodiments related to each individual aspect or specific combinations of these individual aspects.

The above description of exemplary embodiments of the present application has been presented for purposes of illustration and description. It is not intended to be exhaustive or to limit the inventions to the precise forms described, and many modifications and variations are possible in view of the above teachings.

### EXAMPLE 1

An exemplary embodiment of the present application involves using neural measurements to construct a predictive algorithm, which can be used for making outcome forecasting in normal development of language and communication at the individual subject level. For illustration, this exemplary embodiment uses EEG data as the neural measurements. This exemplary embodiment utilizes:
1) an EEG testing protocol that is short and contains auditory stimuli that are most informative;
2) existing data from a group of toddlers who have been tested on this EEG protocol whose future language and communication outcomes are known; and
3) an algorithm to link the EEG and the actual evaluation data of language and communication, that has high specificity and sensitivity in making outcome forecasting.

### Data Source

An exemplary embodiment of the present application utilizes an algorithm constructed based on data from 30 Chinese-learning toddlers. These toddlers underwent EEG neural measurements during infancy and the language developmental outcome was measured using the Chinese Communicative Development Inventories (CCDI, Tardif et al., 2008) several months after the EEG measurements.

### Stimulus and EEG Program

Auditory-evoked EEG procedures are available in literatures (e.g., Lau, J. C. Y., Wong, P. C. M., & Chandrasekaran, B. (2017), “Context-dependent Plasticity in the Subcortical Encoding of Linguistic Pitch Patterns”, Journal of Neurophysiology, 117 (2), 594-603; Liu, F., Maggu, A. R., Lau, J. C. Y., & Wong, P. C. M. (2015), “Brainstem Encoding of Speech and Musical Stimuli in Congenital Amusia: Evidence from Cantonese Speakers”, Frontiers in Human Neuroscience, 8; Maggu, A. R., Liu, F., Antoniou, M., & Wong, P. C. M. (2016), “Neural Correlates of Indicators of Sound Change in Cantonese: Evidence from Cortical and Subcortical Processes,” Frontiers in Human Neuroscience, 10, the contents of which are hereby incorporated by reference in their entirety for all purposes (see FIG. 1 for a basic setup)). According to this exemplary embodiment, infants received auditory stimuli especially crafted for this exemplary embodiment that focused on the property of lexical tone in Chinese (FIG. 2). In the external auditory stimuli, three tones were imbedded into the syllable /ga/: Cantonese tone /ga2/, Mandarin tone /ga3/and Cantonese tone /ga4/. (Note: the infants were being raised in Cantonese-speaking homes by Cantonese-speaking caregivers). The early-latency EEG responses faithfully reflect the time course and the spectral composition of the auditory stimulus. Panel A shows the time courses of the auditory stimuli (left column) and averaged EEG responses (middle column) as well as the averaged spectrogram of the EEG response (right column). The spectral amplitude density of the EEG response concentrates within the upper quartile of the auditory stimulus spectral density. Panel B shows the spectrogram of the auditory stimulus (left column) and averaged EEG spectrograms of the infants at 1, 3, 6, 9 and 15 months of age in response to the auditory stimuli. The older the infants, the clearer is the overlap between the auditory and EEG spectral structures. The collected auditory-evoked neural EEG responses were analyzed following a series of automated procedures implemented in Matlab such as the Fast Fourier Transform, Autocorrelation and Root-Mean-Square (RMS). Measures such as fundamental frequencies (F0s), harmonics, signal-to-noise ratios, and RMS amplitude were extracted following published procedures.

### Other Variables

In addition to neural measurement differences, other variables may also affect language development (e.g., gender, or birth data). Thus, these variables can be entered into the predictive model for optimal predictive performance.

### Machine Learning

The algorithm in this exemplary embodiment uses neural EEG measures for classifying individual infant into better or poorer language outcome groups several months after the EEG recording to be taken during infancy. In this exemplary embodiment, a support vector machine procedure with 10-fold cross-validation and bootstrap-permutation statistics is used for achieving binary classification.

According to this exemplary embodiment, a two-step SVM classification was performed, i.e., at the level of individual subject and at the level of the group of 88 subjects. Out of that group 30 subjects had both language and communication outcome data (CCDI) from the end of their first year of life and EEG data recorded half a year earlier. EEG data were used to forecast the language and communication outcomes six months later.

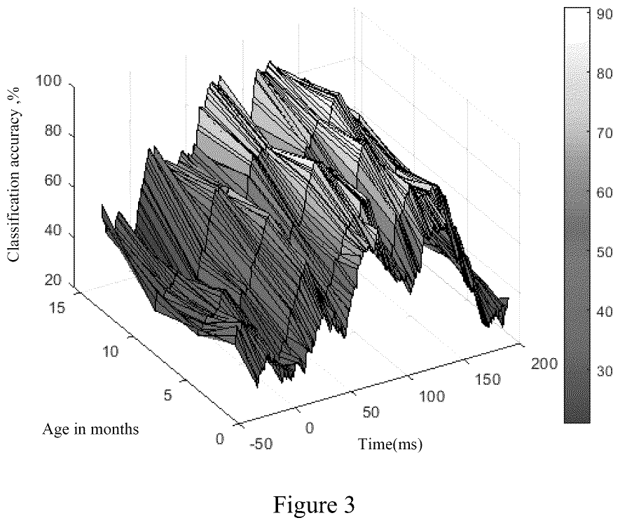

At the level of the individual subject, the filtered EEG was segmented by stimulus onset markers. That resulted in a set of 1000 EEG segments for each of three tones. Each segment was transformed to frequency domain with FFT in a sliding window of 50 ms with 50% overlap between the windows. That resulted in a T*(*3)*F array matrix for each subject, where T is the number of the time windows, E is the number of segments for each tone and F is the number of frequency bins from the FFT analysis. The (E*3)*F matrix for each T was normalized first within rows and then within column thus removing an effect of the absolute amplitude of the spectrum and leaving only the frequency-dependent patterns. Then, the T matrices were fed into a 3-way SVM classifier implemented in LIBSVM software package as predictor for the tone category. Gaussian kernel and C=100 and gamma=1/F were used. The classification was cross-validated with a 10-fold way. That is, the data were randomly divided into 10 parts and for each part the classifier was trained on the 90% of the data and tested on the 10% of the data. The outcome of the cross-validation procedure was the mean accuracy across the folds. As a result, for each subject, a vector of T classification accuracies was obtained. FIG. 3 shows that, by application of the support vector machine (SVM) classifier to the EEG responses of the infants at different months of age to the three Chinese lexical tones /ga2/ /ga3/and /ga4/ to generate forecasted classification outcomes, ability of the infant brain to discriminate the tones can be measured. The SVM classifier was fed with the Fourier transforms of the consequent 50-ms intervals of the along the time course of the EEG data response to the three tones. The classification was cross-validated by training on the 9/10th of the data and testing on the remaining 1/10th of the data for each of the 10 folds of the randomly split the data. Chance level of classification accuracy for 3 tones is 33%. The figure illustrates that the classification accuracy has two peaks along the time course of the EEG response and that the accuracy grows with the age of the infant. The current figure illustrates classification across subjects. The same procedure was performed within each individual subject.

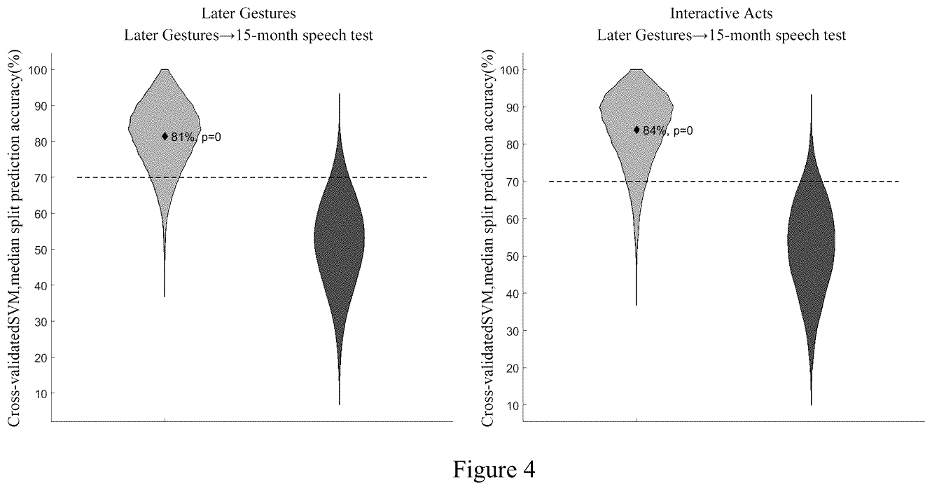

At the group level, those vectors were combined into S*T matrices where S are number the subjects that had both EEG recordings and language and communication outcomes from CCDI. These matrices were created for every possible combination of the age of the EEG and the age of the language and communication outcomes and normalized within each time bin. Those combinations covered partially overlapping subsets of the original 83 subjects. The language and communication outcomes within each of those age combinations were converted into binary variables with median split. Each predictor matrix was normalized within each time bin. Then, binary SVM was performed for each language and communication outcome and for each possible combination of the age of the language and communication outcome and the age of the EEG recording. The SVM parameters were gaussian kernel, C=100 and gamma=1/T. The accuracy of classification was cross-validated with 10-fold procedure. The confidence interval for each classification was calculated with a combination of the bootstrapping and permutation. For each predictor-response combination, the rows of the matrix (subjects) were resampled with replacement, SVM classification with 10-fold cross-validation was performed for both actual and randomly permuted subject labels. The procedure was repeated 10000 times resulting in the distributions of the real and permuted forecasting accuracies (see FIG. 4). The accuracy was considered significantly higher than chance if the mean of the real-label accuracy was higher than the 95th percentile of the distribution of the permuted-label accuracy.

### EXAMPLE 2

On the basis of Example 1, another exemplary embodiment of the present application expanded the toddler cohort to 118 toddlers including the toddler cohort in Example 1 (the experimental protocol was unchanged). The results of the exemplary embodiment using the expanded toddler cohort indicated that the predictive model constructed in the present application could classify toddler as below or above mean levels, or below or above 25% percent, and achieved up to 0.92 accuracy which is measured from Area Under Curve (AUC).

1. A method of forecasting a normal developmental difference or an impairment of language and communication capability of an infant or toddler, comprising:
obtaining, by a computer system, electroencephalogram (EEG) or magnetoencephalogram (MEG) waveform data from the infant or toddler in response to an external auditory stimulus, wherein the external auditory stimulus is from a language and speech signal in which pitch patterns are used to convey meaning at a vocabulary, lexical, phrasal, or sentential level;
extracting, by the computer system, quantitative data from the EEG or MEG waveform data, wherein the quantitative data include measurement index data characterizing a central nervous system response to the external auditory stimulus, wherein the central nervous system includes brainstem;
analyzing, by the computer system, the quantitative data using a machine learning classifier, wherein the machine learning classifier has been trained to provide a forecasting score for forecasting language and communication capability of the infant or toddler relative to a population to which the infant or toddler belongs, and wherein the training is based on corresponding quantitative data from a training data set obtained from a plurality of infants or toddlers known to have normal development of language and communication capability; and
generating, by the computer system, based on an output from the machine learning classifier, a forecasting score for the infant or toddler, wherein the forecasting score is usable for providing intervention or training to the infant or toddler based on the forecasting score.

2. The method of claim 1, further comprising:
obtaining EEG or MEG waveform data of the infant or toddler at rest; and
extracting corresponding quantitative data from the EEG or MEG waveform data of the infant or toddler at rest, wherein the quantitative data comprises measurement index data of the central nervous system at rest.

3. The method of claim 2, wherein the external auditory stimulus comprises a plurality of different external auditory stimuli, and the quantitative data includes measurement index data of the central nervous system response to one or more of the plurality of different external auditory stimuli and measurement index data of the central nervous system at rest.

4. The method of claim 1, wherein the quantitative data includes data characterizing functional activity of the processing pathway associated with the auditory center.

5. The method of claim 1, wherein the quantitative data includes data characterizing functional activity of the inferior colliculus or centers connected to the inferior colliculus.

6. The method of claim 1, wherein the quantitative data includes data characterizing functional activity of the primary auditory cortex, including the Heschl's Gyrus, or centers connected to the Heschl's Gyrus.

7. The method of claim 1, wherein the external auditory stimulus is from Chinese.

8. The method of claim 1, wherein the external auditory stimulus is from English, French, German, Spanish, Portuguese, Japanese, or Korean.

9. The method of claim 7, wherein the external auditory stimulus is a Chinese pinyin with one or more tones.

10. The method of claim 1, wherein the EEG or MEG is performed while the infant or toddler is in sleep or in awake state.

11. The method of claim 1, wherein the infant or toddler has an age of 18 months or less, and wherein when the training data set was obtained from infants or toddlers at ages of 18 months or less.

12. The method of claim 1, wherein the machine learning classifier is a support vector machine (SVM); and
wherein the forecasted language and communication capability of the infant or toddler is a high-degree or a low-degree assessment, or a continuous performance assessment.

13. The method of claim 1, wherein the machine learning classifier is a support vector regression algorithm (SVR) or ranking support vector machine (RankSVM); and
wherein the forecasted language and communication capability of the infant or toddler is quantified.

14. (canceled)

15. The method of claim 1, wherein the quantitative data is extracted from the EEG or MEG waveform data based on: Automatic peak detection, Fast Fourier Transform, Autocorrelation, Root-Mean-Square (RMS), Morlet Wavelet Transform, Discrete Wavelet Transform, Wavelet Scattering, Stimulus-Response Cross-correlation, Empirical Mode Decomposition, or Hilbert-Huang Transform; and/or
wherein the quantitative data includes one or more of: time-domain peak amplitude, time-domain peak latency, fundamental frequency (F0), harmonics, signal-to-noise ratio, RMS amplitude, correlation coefficient, inter-trial phase coherence, phase-locking coefficient, response consistency, pitch strength, pitch error, or pitch-tracking accuracy.

16. The method of claim 1, wherein extracting quantitative data from the EEG waveform data comprises:
segmenting the EEG waveform data by stimulus onset markers, and
transforming each segment to frequency domain with Fast Fourier Transform (FFT) in a sliding time window with an overlap between the windows.

17. The method of claim 16, wherein the quantitative data includes a matrix represented by T*(E*3)*F array for the infant or toddler, wherein T is the number of the time windows, E is the number of segments for each stimulus and F is the number of frequency bins from the FFT analysis, and wherein the (E*3)*F matrix for each T is normalized first within rows and then within columns, thereby removing an effect of the absolute amplitude of the spectrum and leaving only the frequency-dependent patterns over time.

18. The method of claim 17, wherein the machine learning classifier is a support vector machine (SVM) using parameters including Gaussian kernel, C and gamma.

19. The method of claim 17, wherein a classification made by the machine learning classifier is subjected to cross-validation, and the outcome of the cross-validation is average accuracy, specificity, sensitivity, Area Under Curve (AUC), parity rate, correlation coefficient or a combination thereof across certain folds.

20. A non-transitory computer-readable medium storing a plurality of instructions that, when executed by a processor of a computer system, control the computer system to perform operations including:
obtaining electroencephalogram (EEG) or magnetoencephalogram (MEG) waveform data from an infant or toddler in response to an external auditory stimulus, wherein the external auditory stimulus is from a language and speech signal in which pitch patterns are used to convey meaning at a vocabulary, lexical, phrasal or sentential level;
extracting quantitative data from the EEG or MEG waveform data, wherein the quantitative data include measurement index data characterizing a central nervous system response to the external auditory stimulus, wherein the central nervous system includes brainstem;
analyzing the quantitative data using a machine learning classifier, wherein the machine learning classifier has been trained to provide a forecasting score for forecasting language and communication capability of the infant or toddler relative to a population to which the infant or toddler belongs, and wherein the training is based on corresponding quantitative data from a training data set obtained from a plurality of infants or toddlers known to have normal development of language and communication capability; and
generating, based on an output from the machine learning classifier, a forecasting score for the infant or toddler, wherein the forecasting score is usable for providing intervention or training to the infant or toddler based on the forecasting score.

21-38. (canceled)

39. A computer system comprising:
a non-transitory memory have instructions stored thereon; and
one or more processors for executing the instructions stored on the non-transitory memory to facilitate the following being performed by the computer system:
obtaining electroencephalogram (EEG) or magnetoencephalogram (MEG) waveform data from an infant or toddler in response to an external auditory stimulus, wherein the external auditory stimulus is from a language and speech signal in which pitch patterns are used to convey meaning at a vocabulary, lexical, phrasal or sentential level;
extracting quantitative data from the EEG or MEG waveform data, wherein the quantitative data include measurement index data characterizing a central nervous system response to the external auditory stimulus, wherein the central nervous system includes brainstem;
analyzing the quantitative data using a machine learning classifier, wherein the machine learning classifier has been trained to provide a forecasting score for forecasting language and communication capability of the infant or toddler relative to a population to which the infant or toddler belongs, and wherein the training is based on corresponding quantitative data from a training data set obtained from a plurality of infants or toddlers known to have normal development of language and communication capability; and
generating, based on an output from the machine learning classifier, a forecasting score for the infant or toddler, wherein the forecasting score is usable for providing intervention or training to the infant or toddler based on the forecasting score.

40-78. (canceled)
